(19) United States
(12) Patent Application Publication
(10) Pub. No.: US 2019/0224195 A1
(43) Pub. Date: Jul. 25, 2019

(54) Oral Capsule Composite Formulation of Dutasteride and Tadalafil

(71) Applicant: Yuyu Pharma, Inc., Jecheon-si, Chungcheongbuk-do (KR)

(72) Inventors: Taegon Baik, Seoul (KR); Seyeon Kim, Suwon-si, Gyeonggi-do (KR); Ju-Hee Kim, Suwon-si, Gyeonggi-do (KR); Seung Han Song, Suwon-si, Gyeonggi-do (KR); Young-Joon Park, Gwacheon-si, Gyeonggi-do (KR); Kyung Mi Park, Seoul (KR)

(21) Appl. No.: 16/336,971

(22) PCT Filed: Sep. 27, 2017
(86) PCT No.: PCT/KR2017/010684

§ 371 Date: Mar. 27, 2019

(30) Foreign Application Priority Data
Sep. 30, 2016 (KR) 10-2016-0126772

(51) Int. Cl. A61K 31/4985; A61K 31/473; A61K 9/48

(52) U.S. Cl. CPC A61K 31/4985; A61K 31/473; A61P 13/02; A61K 9/4866; A61K 9/4858

(57) ABSTRACT

The present invention relates to a capsule composite formulation comprising dutasteride represented by formula I, tadalafil represented by formula II, a fatty acid ester derivative having 8 to 10 carbon atoms, and a polysorbate- or polyoxylglyceride-based surfactant wherein both the two drugs are dissolved.

### TECHNICAL FIELD

The present invention relates to an oral capsule composite formulation comprising dutasteride and tadalafil, which are poorly-soluble drugs, a fatty acid ester derivative having 8 to 10 carbon atoms as an oil ingredient, and a surfactant. The present invention relates to the composite formulation which increases solubility of both dutasteride and tadalafil, which are poorly-soluble drugs, thereby enhancing dissolution rates of them.

The present invention relates to transparent self-emulsifying drug delivery system-based capsule formulations in which dutasteride and tadalafil are in combination so as to maximize the therapeutic effect on prostatic hyperplasia and which can form emulsions in a self-emulsifying manner so as to rapidly dissolve the two poorly soluble drugs. In the formulation, the self-emulsifying drug delivery system comprises dutasteride, tadalafil, an oil ingredient, and a surfactant, and, if necessary, a solvent. The oil ingredient is a fatty acid ester derivative having 8 to 10 carbon atoms and can improve the solubility of both dutasteride and tadalafil. The surfactant may be based on polysorbate or polyoxylglyceride that provides good solubility for dutasteride and tadalafil. The mixed weight ratio of the oil to the surfactant may be 95:5 to 70:30 to formulate a transparent, self-emulsifying drug delivery system. By means of this system, the solubility of dutasteride and tadalafil can be improved and thus the dissolution rates can also be enhanced.

### BACKGROUND ART

Prostate is a male reproductive organ. The enlargement of the prostate gland is fairly common in older men and more likely to develop with advancing age, particularly after age of 40. An enlarged prostate increases urethral resistance which may result in voiding dysfunction, called benign prostatic hyperplasia. The main cause of prostatic hyperplasia is changes in male sex hormone, testosterone that comes with the aging. It is known that in the old age, testosterone levels are lowered, but dihydrotestosterone (DHT), a metabolite of testosterone, causes enlargement of the prostate.

Representative drugs for prostatic hyperplasia include a 5-alpha reductase inhibitor and a phosphodiesterase (PDE) 5 inhibitor.

U.S. Pat. No. 5,565,467 discloses that dutasteride, a 5-alpha reductase inhibitor, represented by formula I (IUPAC name: 17β-N-(2,5-bis(trifluoromethyl))phenylcarbomoyl-4-aza-5α-androst-1-en-3-one) can be used for the treatment of benign prostatic hyperplasia, prostate cancer, and male alopecia. The 5-alpha reductase inhibitor prevents the conversion of testosterone to dihydrotestosterone (DHT), thereby reducing DHT and inhibiting prostate growth.

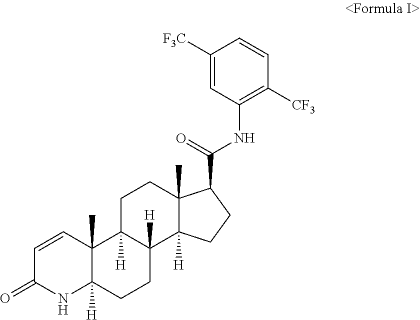

Dutasteride, which is poorly water-soluble, is commercially available as AVODART® 0.5 mg Soft Capsule. AVODART® is a product in the form of a soft capsule wherein 0.5 mg of dutasteride is dissolved in 349.5 mg of a mixture of mono- and diglyceride of caprylic/capric acid and butyl hydroxytoluene.

Tadalafil (chemical name: 6R-trans-6-(1,3-benzodioxol-5-yl)-2,3,6,7,12,12a-hexahydro-2-methyl-pyrazino [1′,2′:1,6]pyrido[3,4-b]indole-1,4-dione), the PDE 5 inhibitor, represented by formula II, was developed as a drug for the treatment of sexual dysfunction, but is used for the treatment of prostatic hyperplasia for daily administration only in the case of the dose of 5 mg.

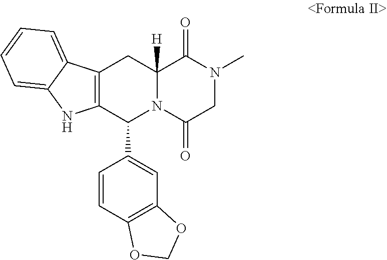 [SMILES: CN1CC(=O)N2[C@H](c3ccc4c(c3)OCO4)c3[nH]c4ccccc4c3C[C@@H]2C1=O]

A combination therapy of the two drug molecules is recommended as a more effective treatment option for moderate to severe urinary tract symptoms than monotherapy.

However, both dutasteride and tadalafil are poorly soluble drugs. In addition, dutasteride is commercially available in the form of a soft capsule formulation (AVODART® 0.5 mg Soft Capsule) and tadalafil is commercially available in the form of a pill (CIALIS® 5 mg Pill). The formulations of the two drugs differ from each other. Thus, there is a need for developing a composite formulation which can contain the two different drugs together, and simultaneously, which can increase the solubility of the two poorly-soluble drugs.

So, the present inventors conducted a research to develop a composite formulation wherein the solubility of the two poorly soluble drugs, dutasteride and tadalafil is improved, wherein the dissolution of two drugs may be completed within 30 minutes, and which can be easily prepared. As a result, the inventors have found that a fatty acid ester derivatives having 8 to 10 carbon atoms as oil ingredients and polysorbates or polyoxylglycerides as surfactants have to be in mixture to prepare a transparent, self-emulsifying drug delivery system-based composite formulation.

### DISCLOSURE OF INVENTION

### Technical Problem

Embodiments of the present invention is directed to provide a self-emulsifying drug delivery system-based composite formulation comprising dutasteride and tadalafil improved in initial dissolution and overall dissolution rate for rapid onset of action and in which two drugs are in the solubilized state in capsules.

### Solution to Problem

A composite formulation according to one embodiment of the present invention is a transparent self-emulsifying drug delivery system-based composite formulation comprising dutasteride and tadalafil represented by formulas I and II, a fatty acid ester derivative having 8 to 10 carbon atoms, and a surfactant:

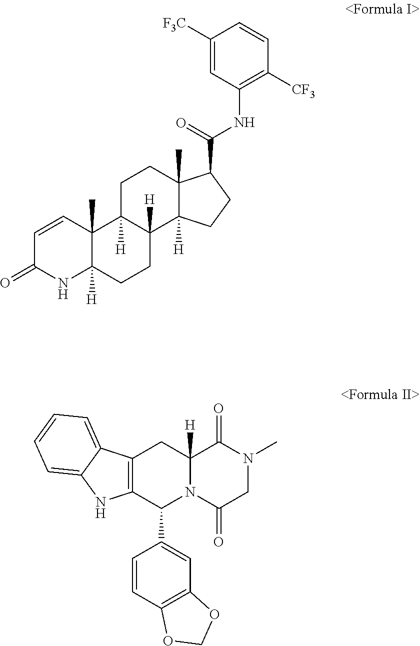

The capsule formulation according to another embodiment of the present invention comprises the transparent, self-emulsifying drug delivery system-based composite formulation.

The foregoing is illustrative only and is not intended to be in any way limiting. In addition to the illustrative aspects, embodiments, and features described above, further aspects, embodiments, and features will become apparent by reference to the drawings and the following detailed description.

### Advantageous Effects of Invention

Embodiment of the present invention can provide a transparent, self-emulsifying drug delivery system-based composite formulation that completely dissolves the poorly soluble drugs, dutasteride and tadalafil, to improve the dissolution rate thereof.

### BRIEF DESCRIPTION OF DRAWINGS

FIG. 1 is a photographic image showing contents of a composite formulation prepared according to an embodiment of the present invention.

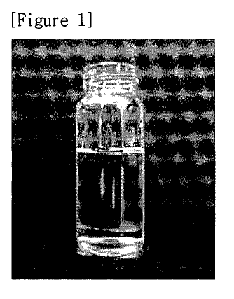

FIG. 2 is a graph showing dissolution test results compared between formulations of the Examples and the Comparative Examples.

### MODE FOR THE INVENTION

The self-emulsifying drug delivery system-based composite preparation according to the present invention includes dutasteride of Formula I and tadalafil of Formula II, an oil composed of a fatty acid ester derivative having 8 to 10 carbon atoms, and a polysorbate- or a polyoxylglyceride-based surfactant, and further a polyoxyethylene as a solvent, if needed.

The pharmaceutically active ingredients dutasteride and tadalafil in the self-emulsifying drug delivery system-based composite preparation are both poorly soluble drugs and can be increased in solubility by use of a fatty acid ester derivative having 8 to 10 carbon atoms and a polysorbate- or polyoxylglyceride-based surfactant in a self-emulsifying drug delivery systems. As an oil ingredient in a self-emulsifying drug delivery system, the fatty acid ester derivative having 8 to 10 carbon atoms can greatly increase the solubility of the main ingredients, dutasteride and tadalafil, and can completely dissolve the poorly water soluble drugs and results a clear appearance. Examples of the fatty acid ester derivative having 8 to 10 carbon atoms include glycerol caprylate/caprate and propylene glycol monocaprylate. These oils provide far higher solubility for the two drugs than other oils, as evaluated in Test Example 1. In addition, it can be verified that the content in the composite preparation has a transparent property as dutasteride and tadalafil are both completely dissolved.

Test Example 1 shows solubility of dutasteride and tadalafil in various kinds of oils. As understood from the data of Table 5, dutasteride and tadalafil are unlikely to be dissolved in castor oil, soybean oil, and polyoxyl 6 apricot kernel oil, but the fatty acid ester derivatives having 8 to 10 carbon atoms exhibit at least 10-fold greater solubility for dutasteride and 4-fold greater for tadalafil.

Based on the solubility test results, a fatty acid ester derivative is used as an ingredient for improving poorly soluble drugs in a self-emulsifying drug delivery system so that dutasteride and tadalafil can be completely dissolved to form a transparent composite preparation. The oil may be particularly used in an amount of 70-95% by weight, based on the total weight of the preparation. For instance, when the oil is used in an amount greater than 95% by weight, self-emulsification is difficult. On the other hand, when the amount of the oil is less than 70% by weight, other ingredients may harden the capsule film to delay the disintegration of the capsule.

In the self-emulsifying drug delivery system-based composite preparation, the surfactant acts to stably emulsify the oil ingredient in water to form an emulsion. Preferable are surfactants that make dutasteride and tadalafil transparent when mixed with an oil ingredient and form nano-emulsions when come in contact with water. Among others, a surfactant that exhibits high solubility for the two drugs and can formulate a self-emulsifying system with a fatty acid ester derivative having 8 to 10 carbon atoms is most preferable. Examples of the surfactant include polysorbates, such as oxyl sorbitan fatty acid esters, i.e., polysorbate 20, polysorbate 40, polysorbate 60, and polysorbate 80, and polyoxylglycerides such as PEG 6 glyceryl caprylate/caprate (Acconon CC-6™), PEG 8 caprylic/capric glyceride (Labrasol™). The surfactants introduced in the related patent (Korean Patent Publication No. 10-2016-0023962), such as polyoxylstearate, polyoxyl castor oil, poloxamer, etc., are difficult to apply to the formation of transparent capsules due to their low solubility for dutasteride and tadalafil. Thus, such surfactants are different from those of the present invention and cannot be used in the present invention. In other words, the surfactant suitable for the purpose of the present invention may include at least one of polysorbates and polyoxylglycerides. The oil and the surfactant may be preferably used at a weight ratio of 95:5 to 70:30.

In this regard, the study on phase equilibrium, conducted by the present inventors, exhibited that a mixture of the surfactant and the oil ingredient propyleneglycol monocaprylate are mixed with each other to form emulsions over a stable and wide area in water.

Since less than 5% of the surfactant brings about low solubility for dutasteride and tadalafil, it is impossible to manufacture the resulting preparation into a capsule formulation having sizes suitable for administration. In addition, when the surfactant is used in an amount greater than 30%, the surfactant may act to harden the capsule film, deteriorating the quality, such as stability, of the drug.

Further, an auxiliary solubilizer such as polyethylene glycol may be used in the capsule to enhance the solubilization of the two drugs. In addition, the preparation of the present invention may further comprise a pharmaceutically acceptable additive for oral administration, for example, antioxidants, and preferably butylhydroxytoluene.

Also, an embodiment of the present invention provides a method for preparing a self-emulsifying drug delivery system-based composition, comprising the steps of mixing dutasteride and tadalafil at a predetermine weight ratio with a fatty acid ester derivative having 8 to 10 carbon atoms and a polysorbate- or polyoxylglyceride-based surfactant and dissolving the dutasteride and the tadalafil. According to the method, dutasteride and tadalafil are mixed with a fatty acid ester derivative and a surfactant to obtain a transparent liquid.

Another embodiment of the present invention provides an oral capsule formulation filled with a self-emulsifying drug delivery system-based preparation comprising dutasteride and tadalafil, an oil ingredient, and a surfactant. Using an automatic rotary capsule loader, the preparation may be encapsulated into a capsule which is made of gelatin, succinylated gelatin, and a plasticizer (glycerin, sorbitol), to produce a soft capsule formulation.

Further, a capsule formulation may be produced by loading the self-emulsifying drug delivery system-based composite preparation into a hard capsule by use of a hard capsule filling machine for liquid filling.

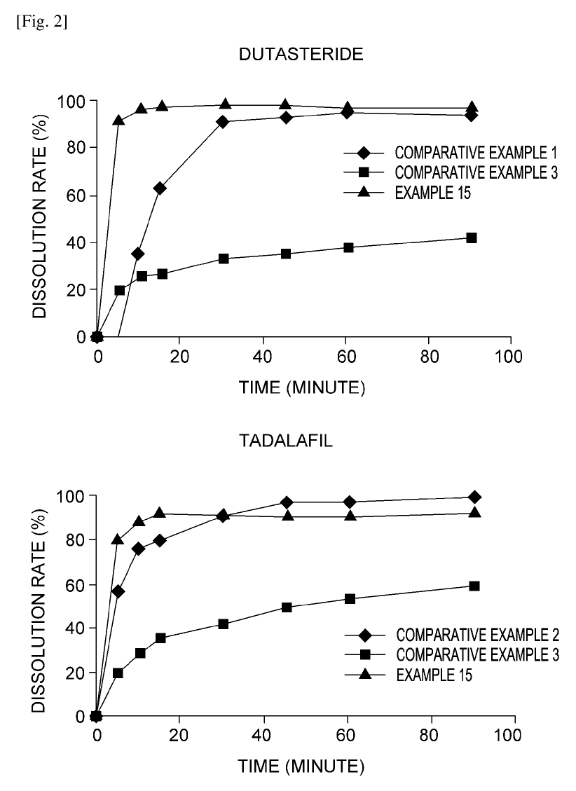

The oral capsule formulation according to some embodiments includes a self-emulsifying drug delivery system-based composite preparation in which dutasteride and tadalafil are dissolved at enhanced solubility and guarantees enhanced dissolution rates for the drugs than the soft capsule AVODART and the commercially available CIALIS pill. With reference to FIG. 2, the self-emulsifying drug delivery system-based composite preparation was observed to exhibit higher dissolution rates than the Comparative Examples.

Hereinafter, the composite formulation according to embodiments of the present invention will be described in detail with reference to examples and comparative examples. However, this does not limit the scope of the present invention.

### Examples 1-8

To a 5-L preparation container equipped with a stirrer, oils and surfactants were added in amounts as shown in Table 2, below, and the mixture was stirred during which 0.5 g of dutasteride was slowly added and completely dissolved. Then, 0.5 g of tadalafil was added and completely dissolved. The resulting mixture was further stirred, together with 0.1 g of butylhydroxy toluene, to form a transparent self-emulsifying drug delivery system-based composite preparation. Separately, a soft gelatin capsule film was prepared using typical gelatin, a plasticizer and the like, as shown in Table 1. The solubilizing preparation was filled into the soft capsule to produce a soft capsule formation.

TABLE 1

| Ingredient | wt. % |
|---|---|
| Succinylated gelatin | 71.17 |
| Concentrated glycerin | 20.16 |
| Disorbitol solution | 8.67 |
| Total | 100% |

TABLE 2

| | | Example No. | | | | | | | | | | | | | | | |
|---|---|---|---|---|---|---|---|---|---|---|---|---|---|---|---|---|---|
| Class | Ingredient | 1 | | 2 | | 3 | | 4 | | 5 | | 6 | | 7 | | 8 | |
| API | Dutasteride | 0.5 | g | 0.5 | g | 0.5 | g | 0.5 | g | 0.5 | g | 0.5 | g | 0.5 | g | 0.5 | g |
| | Tadalafil | 5 | g | 5 | g | 5 | g | 5 | g | 5 | g | 5 | g | 5 | g | 5 | g |
| Oil | Glyceryl caprylate/caprate | 960 | g | 1080 | g | 1080 | g | 1080 | g | 1080 | g | 1080 | g | 1080 | g | 1178 | g |
| Surfactant | Polysorbate 60 | | | 360 | g | 120 | g | | | | | 120 | g | | | | |
| | Polysorbate 80 | | | | | 240 | g | | | | | | | 120 | g | | |
| | PEG 8 caprylic/capric glyceride | 360 | g | | | | | 240 | g | 360 | g | 120 | g | 120 | g | 62 | g |
| Antioxidant | Butylhydroxytoluene | 0.13 | g | 0.14 | g | 0.14 | g | 0.13 | g | 0.14 | g | 0.12 | g | 0.13 | g | 0.12 | g |

### Examples 9-16

To a 5-L preparation container equipped with a stirrer, oils and surfactants were added in amounts as shown in Table 3, below, and the mixture was stirred during which 0.5 g of dutasteride was slowly added and completely dissolved. Then, 5 g of tadalafil was added and completely dissolved. The resulting mixture was further stirred, together with 0.1 g of butylhydroxy toluene, to form a transparent self-emulsifying drug delivery system-based composite preparation. Separately, a soft gelatin capsule film was prepared using typical gelatin, a plasticizer and the like, as shown in Table 1. The solubilizing preparation was filled into the soft capsule to produce a soft capsule formation.

TABLE 3

| | | Example No. | | | | | | | | | | | | | | | |
|---|---|---|---|---|---|---|---|---|---|---|---|---|---|---|---|---|---|
| Class | Ingredient | 9 | | 10 | | 11 | | 12 | | 13 | | 14 | | 15 | | 16 | |
| API | Dutasteride | 0.5 | g | 0.5 | g | 0.5 | g | 0.5 | g | 0.5 | g | 0.5 | g | 0.5 | g | 0.5 | g |
| | Tadalafil | 5 | g | 5 | g | 5 | g | 5 | g | 5 | g | 5 | g | 5 | g | 5 | g |
| Oil | Propylene glycol monocaprylate | 850 | g | 960 | g | 960 | g | 1000 | g | 1000 | g | 1080 | g | 1080 | g | 1190 | g |
| Surfactant | Polysorbate 60 | | | 360 | g | | | 240 | g | 360 | g | | | | | | |
| | Polysorbate 80 | | | | | 240 | g | | | | | | | 120 | g | | |
| | PEG 8 caprylic/capric glyceride | 360 | g | | | 120 | g | | | | | | | 240 | g | 60 | g |
| Antioxidant | Butylhydroxytoluene | 0.12 | g | 0.13 | g | 0.13 | g | 0.12 | g | 0.14 | g | 0.12 | g | 0.13 | g | 0.12 | g |

### Comparative Example 1

The commercial product AVODART® 0.5 mg soft capsule, which corresponds to 0.5 mg of dutasteride, was used.

### Comparative Example 2

The commercial product CIALIS® 5 mg pill, which corresponds to 5 mg of tadalafil, was used.

### Comparative Example 3

To a 5-L preparation container, the oil was added in an amount as shown in Table 4, below. Following slowly adding and completely dissolving 0.5 g of dutasteride, 5 g of tadalafil was added and completely dissolved. The resulting composition was filled to a hard capsule to prepare an oral hard capsule formulation.

TABLE 4

| Class | Ingredient Name | COMPARATIVE EXAMPLE 3 |
|---|---|---|
| API | Dutasteride | 0.5 g |
| | Tadalafil | 5 g |
| Oil | Glycerylmonooleate | 156 g |
| Surfactant | Kolliphor RH 40 | 4.95 g |
| | Poloxamer 124 | 3.3 g |

### Test Example 1: Solubility Test

Dutasteride and tadalafil were measured for solubility in various oils comprising soybean oil, castor oil, polyoxyl 6 apricot kernel oil, propyleneglycol monolaurate, propyleneglycol monocaprylate, glyceryl caprylate/caprate. In a 10-mL vial, 3 mL of an oil was stirred at room temperature with a magnetic bar during which about 100 mg of the main ingredient was added, followed by stirring at 500 rpm or higher. After 24 hours of stirring, centrifugation was carried out and the supernatant thus formed was taken and subjected to liquid chromatography to quantitate the main ingredient dissolved in the oil phase.

TABLE 5
Solubility of Dutasteride and Tadalafil According to Solvent

| | Solubility | |
|---|---|---|
| | of Dutasteride | Solubility of |
| Solvent | (mg/mL) | Tadalafil (mg/mL) |
| Soybean oil | 0.00 | 0.24 |
| Castor oil | 1.50 | 1.03 |
| Polyoxyl 6 apricot kernel oil | 2.55 | 1.12 |
| Propylene glycol monocaprylate | 26.27 | 4.61 |
| Glyceryl caprylate/caprate | 14.34 | 4.13 |

Solubility of the main ingredients in various oils is given in Table 5. As is understood from the solubility result shown in Table 5, the fatty acid ester derivatives glyceryl caprylate/caprate and propylene glycol monocaprylate exhibit 10- or more fold greater solubility for dutasteride and 4- or more fold greater solubility for tadalafil than the other oils.

TABLE 6
Solubility of Dutasteride and Tadalafil According to Surfactant

| | | Tadalafil |
|---|---|---|
| | Dutasteride | Solubility |
| Surfactant | Solubility (mg/mL) | (mg/mL) |
| PEG 35 castor oil | 1.99 | 8.1 |
| Polysorbate 60 | 1.77 | 10.74 |
| Polysorbate 80 | 1.36 | 9.65 |
| PEG 6 glyceryl carpylate/caprate | 3.55 | 9.13 |
| PEG 8 caprylic/capric glyceride | 3.86 | 8.81 |
| Poloxamer 124 | 1.12 | 6.23 |
| Diethylene glycol monoethyl ether | 0 | 0 |

Test results of solubility according to surfactants are summarized in Table 6. As seen, the polyoxylglycerides PEG 6 glyceryl caprylate/caprate, and PEG 8 caprylic/capric glyceride ensured high solubility for dutasteride and tadalafil.

### Test Example 2: Dissolution Test

The soft capsule of Example 15, the commercially available formulations AVODART® 0.5 mg soft capsule and CIALIS® 5 mg pill, respectively used in Comparative Examples 1 and 2, and the hard capsule of Comparative Example 3 were subjected to the dissolution test of Comparative Example 3. The dissolution test was conducted according to Korean Pharmacopeia Dissolution Apparatus 2, in which an aqueous 1% lauryl sodium sulfate solution was used as a dissolution medium and a stirring speed was set to be 50 rpm.

As seen in FIG. 2, the oral soft capsule formulation used in the Example of the present invention enhanced solubility of the poorly soluble drugs, exhibiting higher dissolution rates than AVODART of Comparative Example 1, CIALIS Comparative Example 2, and the capsule of Comparative Example 3.

1. An oral capsule composite formulation, comprising:
dutasteride, represented by formula I;
tadalafil, represented by formula II;
a surfactant; and
a fatty acid ester derivative having 8 to 10 carbon atoms,
wherein the derivative dissolves both dutasteride and tadalafil:

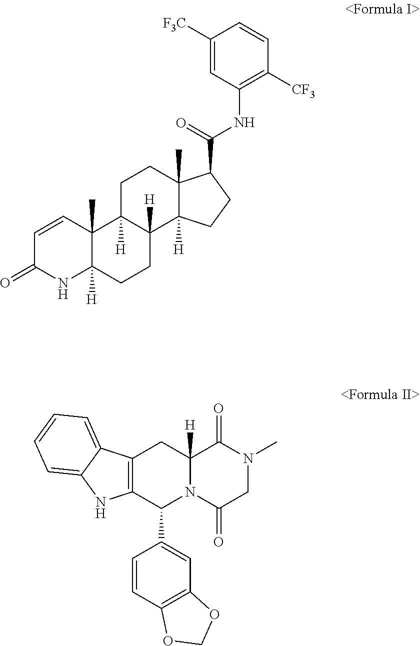

2. The composite formulation of claim 1, wherein the derivative is glyceryl caprylate/caprate or propylene glycol monocaprylate.

3. The composite formulation of claim 1, wherein the surfactant comprises at least one of polyoxyl sorbitan fatty ester and polyoxylglyceride.

4. The composite formulation of claim 1, wherein a content of the fatty acid ester derivative is in a range from 70 to 95%.

5. The composite formulation of claim 1, wherein a mixed weight ratio of the oil to the surfactant is 95:5 to 70:30.

6. The composite formulation of claim 1, comprising 0.5 mg of dutasteride and 5 mg of tadalafil per capsule.

7. The composite formulation of claim 1, which spontaneously forms emulsion in a body after being administered.

8. The composite formulation of claim 1, which has a content filled to a capsule in an amount of 400 to 1500 mg.
